(19) United States
(12) Patent Application Publication
(10) Pub. No.: US 2005/0226826 A1
(43) Pub. Date: Oct. 13, 2005

(54) Delivery system for an active agent

(75) Inventors: Michael Douglas Eason, Bebington (GB); Craig Warren Jones, Bebington (GB); Steven Paul Rannard, Bebington (GB); Brodyck James Lachlan Royles, Bebington (GB); Michael Stephen White, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/099,906

(22) Filed: Apr. 5, 2005

(30) Foreign Application Priority Data
Apr. 8, 2004 (GB) 0408012.3

(51) Int. Cl. A61K 7/44

(52) U.S. Cl. 424/60

(57) ABSTRACT

A delivery system comprising an active agent encapsulated by a polymeric film comprising a polymeric backbone derived from a polymer which is water soluble, and one or more derivatising groups attached to the backbone, the derivatising group(s) being derived from a parent material having a ClogP of from 0.5 to 6, the delivery system also comprising a surfactant on the outside of the polymeric film.

The present invention is concerned with a system and method for the controlled release of an active agent and involves encapsulation and polymer-surfactant interactions. The invention may be used in the controlled delivery of active agents useful in home and personal care.

Encapsulation of an active agent by a polymeric film is known as a method for delaying the release of the active agent into the surrounding environment. Such methods have been used in numerous fields, including those of medicine, agrochemicals, and home and personal care.

Many types of water soluble polymeric films have been used for encapsulation, including polyols such as poly(vinyl alcohol) (hereinafter referred to as “PVOH”).

EP-A-518689 discloses a delivery system for hazardous materials (for example pesticides) comprising a PVOH film encapsulating a composition comprising the hazardous material.

EP-B-389513 discloses concentrated aqueous syrups inside PVOH films, the concentration of the syrup being effective to prevent dissolution of the film.

EP-A-700989 discloses a dish washing detergent composition wrapped in PVOH film, wherein the film protects the detergent from dissolution until the main wash cycle of the dish washing machine.

WO-A-97/27743 discloses an agrochemical composition packaged in a water soluble sachet, which can be PVOH.

GB-A-2118961 discloses bath preparations packaged in PVOH film, while EP-B-347221 relates to water-soluble sachets of phytosanitary materials which are packaged in a secondary water-insoluble pack with a humid environment being maintained between the two.

EP-A-593952 discloses a water soluble sachet of PVOH with two chambers and a treatment agent for washing inside each chamber.

EP-A-941939 relates to a water soluble package, which can be PVOH, containing a composition which, when dissolved, produces a solution of known composition.

EP-B-160254 relates to a washing additive comprising a mixture of detergent constituents in a PVOH bag. The detergent comprises nonionic surfactant and a quaternary ammonium compound.

GB-A-2305931 discloses a dissolvable laundry sachet and BE-9700361 relates to a water soluble unit-dosed cleaning agent, especially for cleaning hands.

DE-29801621 discloses a water soluble unit dose for dishwashing machines.

U.S. Pat. No. 4,846,992 discloses a double-packaged laundry detergent wherein the inner package is water-soluble and can be PVOH.

EP-B-158464 relates to a detergent mull packaged in PVOH and DE-A-19521140 discloses a water soluble PVOH sachet containing a detergent composition.

FR-2601930 relates to a water soluble sachet containing any substance, particularly a pharmaceutical.

A variety of types of water soluble PVOH films are also known. For example, EP-B-157162 relates to a self-supporting film comprising a PVOH matrix having rubbery microdomains dispersed therein.

WO-A-96/00251 relates to an amphipathic graft copolymer comprising a hydrophobic backbone with grafting sites to which are grafted a hydrophilic polymer prepared from a hydrophilic monomer containing stabilising pH independent ionic groups.

GB-B-2090603 relates to a water soluble film comprising a uniform mixture of partially hydrolysed polyvinyl acetate and polyacrylic acid.

WO-A-97/00282 relates to a water soluble film combining two polymeric ingredients S and H where S is a soft acid-functional olefinic addition copolymer having a Tg less than 20° C. and H is a hard acid-functional olefinic addition copolymer having a Tg less than 40° C. The ratio of S:H is from 90:10 to 65:35 and the acid functionalities are at least partially neutralised to render the film water soluble.

EP-B-79712 relates to a laundry additive for discharge to a wash containing borate ions. The additive is enclosed within a film of PVOH which is plasticised and has as a solubiliser either a polyhydroxy compound (such as sorbitol) or an acid (such as polyacrylic acid).

EP-B-291198 relates to a water soluble film containing an alkaline or borate-containing additive. The film is formed from a copolymer resin of vinyl alcohol having 0-10 mole % residual acetate groups and 1-6 mole % of a non-hydrolysable anionic comonomer. FR-2724388 discloses a water soluble bottle, flask or drum made from PVOH which is plasticised with 13-20% of plasticiser (such as glycerol) and then moulded.

The specifications of International Patent Applications WO-A-00/55044, WO-A-00/55045, WO-A-00/55046, WO-A-00/55068, WO-A-00/55069 and WO-A-00/55415 disclose water soluble packages containing a fluid substance (defined as a liquid, gel or paste) which are horizontal form-fill-seal (HFFS) envelopes. These packages comprise a body wall portion having internal volume and which is preferably dome-shaped, formed from a first sheet, and a superposed base wall portion, formed from a second sheet, seded to the body wall portion.

A PVOH package containing a liquid laundry detergent composition comprising from about 10% to about 24% by weight of water (but 3.57% in the sole example) is disclosed in U.S. Pat. No. 4,973,416.

EP0283180 discloses the preparation of very fast dissolving films with a high degree of hydrolysis.

WO-A1-97/19961 discloses fast solubility polymers, made from PVOH co-polymerized with carboxylate moieties, and having some degree of lactonization. These materials dissolve quickly in detergent solution. There is no reference or suggestion to control of solubility using washing surfactants.

EP0284334 relates to films comprising a blend of PVOH and alkyl celluloses with a metal salt, such as borate, to produce a triggered pouch. The alkyl cellulose is present to respond to temperature such that at low rinse temperatures it is more soluble than at the higher temperatures associated with the wash cycle. The borate cross linking provides pH sensitivity. Furthermore, this document discloses that anionic surfactants have very little effect on or even increase the rate of dissolution of the film.

GB2358382 relates to rigid blow molded components made from PVOH.

AT408548 concerns PVOH materials that contain builders for the improvement of detergency during the wash cycle.

When formulating a liquid unit dose product of the kind wherein a substantially non-aqueous formulation is encapsulated in a water soluble film, probably the most difficult challenge is to preserve the physical integrity and stability of the film. One approach to this problem is disclosed in WO-A1-01/79417, which involves substantially neutralising, or over-neutralising any acidic components in the liquid composition, especially any fatty acids and/or acid precursors of anionic surfactant. However, this approach is specific to encapsulation using a water-soluble film based on PVOH which includes co-monomer units having carboxyl functionality.

Preservation of the integrity of films which contain fabric softening compositions for use in the rinse cycle is particularly challenging since commercial softening compositions are generally aqueous and tend to interact undesirably with water soluble packaging causing a weakening of the film and potentially premature breakage, e.g. during storage.

One way of addressing this problem is disclosed in U.S. Pat. No. 4,765,916 which involves providing a cross-linked polymeric water soluble film, preferably a borate.

Where the product is used to deliver a fabric softening composition, it is important that the contents are delivered primarily during the rinse cycle.

In the case of so-called “top-loading” washing machines where the fabric conditioning product is typically dosed directly into the drum of the washing machine, this usually requires that the consumer to be present both at the beginning of the wash cycle and at the beginning of the rinse cycle to dose the wash and rinse products respectively.

Accordingly, it is desirable to be able to provide a product which can be dosed into the washing machine drum at the beginning of the wash cycle but does not disperse or release its contents until the rinse cycle.

One way of addressing this problem is set out in WO-A1-02/102956, where a water soluble package is provided which is soluble in response to, for instance, the change in pH and/or ionic strength from the wash liquor to the rinse liquor. However, the variety of machines and wash conditions means that changes in pH and/or ionic strength can vary enormously. Therefore, it is also desirable to provide a water soluble package which can be dosed into the wash cycle and which is triggered in the rinse cycle by an alternative means.

WO-A-01/85892 discloses highly concentrated conditioners with PVOH film receptacles which are added to the rinse compartment of the dosing drawer. The receptacle enters the rinse bath when the rinse cycle starts.

WO-A-00/51724 discloses the use of molecular sieves for controlled release of fabric treatment products.

WO-A-00/06688 relates to PVOH films which are modified with an amine group. The film releases its contents due to a change in pH during the laundry cycle.

DE-A-2749555 discloses a two fold laminate with a washing pouch, released during the rinse. However, an insoluble bag remains after the laundry cycle is complete. Furthermore, the polymers discloses therein are not hydrophobically modified.

The inventors have now found that the water solubility of a polymeric film comprising a hydrophobically-modified polyol can be modified by adjusting the level of surfactant absorbed upon its surface. This enables a delivery system to be designed in which release of an active agent encapsulated by such a film may be triggered by adjusting, in particular lowering, the level of surfactant absorbed upon the surface of the film.

It has been further found that the level of surfactant absorbed upon the surface of a polymeric film comprising a hydrophobically-modified polyol can be lowered by dilution of the surfactant concentration in the surrounding environment and/or by increasing the temperature. This has enabled the invention of delivery systems suitable for use in the fields of medicine, agrochemicals, and, in particular, home and personal care.

A particular use in the field of home care has been found in the domestic laundry process. It has been found that by hydrophobically modifying the structure of a water soluble polymeric film, such as a PVOH film, with a modifying group, e.g. with one or more acetal groups, the film remains substantially intact in the presence of an external surfactant, e.g. during the wash cycle of a laundry operation, and disintegrates when the concentration of the surfactant reduces sufficiently, e.g. during the rinse cycle of the laundry operation.

According to a first aspect of the present invention there is provided a delivery system comprising an active agent encapsulated by a polymeric film comprising a polymeric backbone derived from a polymer which is water soluble, and one or more derivatising groups attached to the backbone, the derivatising group(s) being derived from a parent material having a ClogP of from 0.5 to 6, the delivery system also comprising a surfactant on the outside of the polymeric film.

According to a second aspect of the present invention there is provided a delivery system comprising an active agent encapsulated by a polymeric film comprising a hydrophobically-modified polyol, the delivery system also comprising a surfactant on the outside of the polymeric film.

According to a third aspect of the present invention there is provided a method of delivering an active agent to a target comprising dilution and/or heating of a delivery system according to the first or second aspect of the invention.

The active agent may be any treatment agent suitable for the task required. The active agent may be a pharmaceutical agent, an agrochemical agent, a cleaning or laundering agent, or a cosmetic agent, such as a perfume or skin care agent.

The active ingredient may be co-encapsulated with one or more carrier materials and/or other components. The state of matter of the total encapsulated material (hereinafter called the “encapsulate”) is preferably liquid. The encapsulate is preferably substantially non-aqueous, such encapsulates being compatible with and reliably protected by the polymeric films as described herein.

In the context of the present invention, “substantially non-aqueous” means that the level of water in the encapsulate is less than 20% by weight of the total weight of the encapsulate, more preferably 15% or less by weight, most preferably 10%, e.g. 5% or even 3% or less by weight.

The level of encapsulate within each delayed release package (vide infra) is typically from 0.5 g to 100 g, in particular from 1 g to 30 g, and especially from 1.5 g to 25 g, e.g. from 2 g to 15 g.

The encapsulate may be a composition suitable for use in home or personal care; for example, it may be a cosmetic composition, a domestic cleaning composition, or a fabric treatment composition such a fabric softening composition. Particularly suitable fabric softening compositions for use in the present invention include substantially non-aqueous melts, emulsions, and microemulsions.

A substantially non-aqueous melt is a fabric softening composition present in solid form, such as particles, at a specified temperature, the solid being suspended in an oil matrix and containing less than 20 wt %, preferably less than 5 wt % of water.

A substantially non-aqueous concentrated rinse conditioner emulsion is a mixture of a quaternary ammonium softening material, an oil and water at a level of less than 20 wt %.

A substantially non-aqueous microemulsion is a composition comprising less than 20% by weight water, wherein the composition is clear, isotropic and thermodynamically stable across a range of temperatures.

Preferred fabric softening compositions used in the present invention are concentrated, meaning that they comprise 10% by weight or greater of fabric softening active agent. More preferred fabric softening compositions are super-concentrated, meaning that they comprise 25% by weight or greater of fabric softening active agent, typically comprising from 25% to 97%, preferably from 35 to 95%, more preferably from 45 to 90%, and most preferably from 55 to 85% by weight of fabric softening active agent.

An encapsulate that is a fabric softening composition comprises a fabric softening active agent. Such an agent may be selected from any of those commonly employed for that purpose. Preferred fabric softening active agents are cationic water insoluble quaternary ammonium compounds comprising two C₁₂₋₁₈ alkyl or alkenyl groups connected to the nitrogen head group via at least one ester link. It is more preferred if the quaternary ammonium compound has two ester links.

Particular cationic fabric softening compounds that may be employed are represented by formula (I): 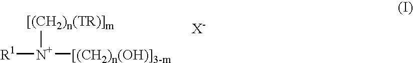
wherein each R is independently selected from a C₅₋₃₅ alkyl or alkenyl group, R¹ represents a C₁₋₄ alkyl, C₂₋₄ alkenyl or a C₁₋₄ hydroxyalkyl group, T is —O—CO.— or —CO.O—, n is 0 or a number selected from 1 to 4, m is 1, 2 or 3 and denotes the number of moieties to which it relates that pend directly from the N atom, and X⁻ is an anionic group, such as halides or alkyl sulphates, e.g. chloride, methyl sulphate or ethyl sulphate.

Especially preferred materials within this class are di-alkenyl esters of triethanol ammonium methyl sulphate. Commercial examples include Tetranyl AHT-1 (di-hardened oleic ester of triethanol ammonium methyl sulphate 80% active), AT-1 (di-oleic ester of triethanol ammonium methyl sulphate 90% active), L5/90 (palm ester of triethanol ammonium methyl sulphate 90% active), all ex Kao, and Rewoquat WE15 (C₁₀-C₂₀ and C₁₆-C₁₈ unsaturated fatty acid reaction products with triethanolamine dimethyl sulphate quaternised 90% active), ex Witco Corporation.

Further cationic fabric softening compounds that may be employed are represented by formula (II): 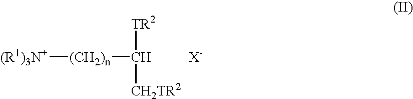
wherein each R¹ group is independently selected from C₁₋₄ alkyl, hydroxyalkyl or C₂₋₄ alkenyl groups; and wherein each R² group is independently selected from C₈₋₂₈ alkyl or alkenyl groups; n is 0 or an integer from 1 to 5 and T and X⁻ are as defined above.

Preferred materials of this class such as 1,2 bis[tallowoyloxy]-3-trimethylammonium propane chloride and 1,2-bis[oleyloxy]-3-trimethylammonium propane chloride and their method of preparation are, for example, described in U.S. Pat. No. 4,137,180 (Lever Brothers), the contents of which are incorporated herein.

Still further cationic fabric softening compounds that may be employed are represented represented by formula (III): 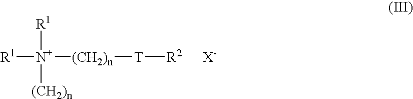
wherein each R¹ group is independently selected from C₁₋₄ alkyl, or C₂₋₄ alkenyl groups; and wherein each R² group is independently selected from C₈₋₂₈ alkyl or alkenyl groups; n is 0 or an integer from 1 to 5 and T and X⁻ are as defined above. A preferred material within this class is N,N-di(tallowoyloxyethyl)-N,N-dimethyl ammonium chloride.

If the quaternary ammonium softening agent comprises hydrocarbyl chains formed from fatty acids or fatty acyl compounds which are unsaturated or at least partially unsaturated (e.g. having an iodine value of from 5 to 140, preferably 5 to 100, more preferably 5 to 60, most preferably 5 to 40, e.g. 5 to 25), then the cis:trans isomer weight ratio of the chains in the fatty acid/fatty acyl compound is greater than 20:80, preferably greater than 30:70, more preferably greater than 40:60, most preferably greater than 50:50, e.g. 70:30 or greater. It is believed that higher cis:trans isomer weight ratios afford the compositions comprising the compound better low temperature stability and minimal odour formation. Suitable fatty acids include Radiacid 406, ex. Fina.

For improved rapid dispersion and/or dissolution of the composition after its release from the polymeric film, it is preferred that the fatty acyl compounds or fatty acids from which the softening compound is formed have an average iodine value of from 5 to 140, more preferably 10 to 100, most preferably 15 to 80, e.g. 25 to 60.

The method for calculating the iodine value is as described in WO-A1-01/04254.

Other components that may be co-encapsulated with the active agent, in particular a fabric softening active agent, include co-actives and formulation and/or dispersion aids.

Co-Actives

Oily sugar derivatives are one form of co-active and may be present in the encapsulate in an amount of from 0.001 to 10 wt %, preferably from 0.01 to 5 wt %, and more preferably from 0.1 to 4 wt %, based on the total weight of the encapsulate. Preferred oily sugar derivatives are those described as CPE's or RSE's in WO-A-96/16538. A particularly preferred oily sugar derivative is a polyester of sucrose.

Oily sugar derivatives may be employed as co-actives in encapsulates that are fabric softening compositions. Other co-actives that may be employed for this purpose are fatty amines and fatty N-oxides. Co-actives in fabric softening composition encapsulates are typically used at from 0.01 to 20% by weight and preferably at from 0.05 to 10%, based on the total weight of the encapsulate.

Formulation and/or Dispersion Aids

Examples of formulation and/or dispersion aids include the following components:
(a) nonionic stabilising agents;
(b) polymeric stabilisers;
(c) single chain cationic surfactants;
(d) fatty alcohols, acids, or oils;
(e) short chain alcohols or oils; or
(f) electrolytes
   a) Nonionic Stabilising Agents.

Suitable nonionic stabilising agents for the encapsulate are nonionic surfactants, as described later as suitable for use as the surfactant used on the outside of the polymer film.

The nonionic stabilising agents may be present in an amount from 0.01 to 10%, preferably from 0.1 to 5%, more preferably from 0.35 to 3.5%, and most preferably from 0.5 to 2% by weight, based on the total weight of the encapsulate.

(b) Polymeric Stabilisers.

Polymeric stabilisers suitable for use preferably comprise at least 2% by weight of water soluble groups either within the main polymer backbone or pendant thereto.

Examples of suitable polymeric materials within this class include PVA; polylactones such as polycaprolactone and polylactide; methyl cellulose; derivativised starches; derivatives of cellulose; and cationic polymers such as Guar Gum.

If present, it is desirable to incorporate such polymers at a level of from 0.01 to 5%, more preferable 0.05 to 3.5%, most preferably from 1 to 2% by weight of the polymer based on the total weight of the composition.

(c) Single Chain Cationic Surfactants.

Single chain cationic surfactants are particularly suitable for use in emulsion encapsulates, since they can be employed to aid the dispersion characteristics of the emulsion.

Suitable single chain cationic surfactants are quaternary ammonium compound comprising a hydrocarbyl chain having 8 to 40 carbon atom, preferably 8 to 30, more preferably 12 to 25 carbon atoms (quaternary ammonium compounds comprising a C₁₀₋₁₈ hydrocarbyl chain are especially preferred).

Examples of commercially available single chain cationic surfactants which may be used include: ETHOQUAD (RTM) 0/12 (oleylbis(2-hydroxyethyl)methylammonium chloride); ETHOQUAD (RTM) C12 (cocobis(2-hydroxyethyl)methyl ammonium chloride) and ETHOQUAD (RTM) C25 polyoxyethylene(15)cocomethylammonium chloride), all ex. Akzo Nobel; SERVAMINE KAC (RTM), (cocotrimethylammonium methosulphate), ex. Condea; REWOQUAT (RTM) CPEM, (coconutalkylpentaethoxymethylammonium methosulphate), ex. Witco; cetyltrimethylammonium chloride (25% solution supplied by Aldrich); RADIAQUAT (RTM) 6460, (coconut oil trimethylammonium chloride), ex. Fina Chemicals; NORAMIUM (RTM) MC50, (oleyltrimethylammonium chloride), ex. Elf Atochem.

The single chain cationic surfactant is preferably present in an amount from 0 to 5% by weight, more preferably 0.01 to 3% by weight, most preferably 0.5 to 2.5% by weight, based on the total weight of the encapsulate.

(d) Fatty Alcohols, Acids, or Oils.

These formulation aids may be selected from fatty alcohols, acids or oils, for example C8 to C₂₋₄ alkyl or alkenyl monocarboxylic acids, alcohols or polymers thereof and C₈ to C₃₅ oils. Preferably saturated fatty acids or alcohols are used, in particular, hardened tallow C₁₆ to C₁₈ fatty acids.

Preferably the fatty acid is non-saponified, more preferably the fatty acid is free, for example oleic acid, lauric acid or tallow fatty acid. The level of fatty acid material is preferably more than 0.1% by weight, more preferably more than 0.2% by weight. Concentrated and superconcentrated compositions may comprise from 0.5 to 20% by weight of fatty acid, more preferably 1% to 10% by weight.

Suitable fatty acids include stearic acid (PRIFAC 2980), myristic acid (PRIFAC 2940), lauric acid (PRIFAC 2920), palmitic acid (PRIFAC 2960), erucic acid (PRIFAC 2990), sunflower fatty acid (PRIFAC 7960), tallow acid (PRIFAC 7920), soybean fatty acid (PRIFAC 7951) all ex. Uniqema; azelaic acid (EMEROX 1110) ex. Henkel.

The fatty acid may also act as a co-softener when used in a fabric softener composition.

Alternatively or additionally the encapsulate may comprise a long chain (ie. “fatty”) oil, typically having 12 carbon atoms or greater. The oil may be a mineral oil, an ester oil, a silicone oil and/or natural oils such as vegetable or essential oils. However, ester oils or mineral oils are preferred.

The ester oils are preferably hydrophobic in nature. They include fatty esters of mono or polyhydric alcohols having from 1 to 24 carbon atoms in the hydrocarbon chain, and mono or polycarboxylic acids having from 1 to 24 carbon atoms in the hydrocarbon chain, provided that the total number of carbon atoms in the ester oil is equal to or greater than 8., and that at least one of the hydrocarbon chains has 12 or more carbon atoms.

Suitable ester oils include saturated ester oils, such as the PRIOLUBES (ex. Uniqema). 2-ethyl hexyl stearate (PRIOLUBE 1545), neopentyl glycol monomerate (PRIOLUBE 2045) and methyl laurate (PRIOLUBE 1415) are particularly preferred although oleic monoglyceride (PRIOLUBE 1407) and neopentyl glycol dioleate (PRIOLUBE 1446) are also suitable.

Suitable mineral oils include branched or straight chain hydrocarbons (e.g. paraffins) having 8 to 35, more preferably 9 to 20 carbon atoms in the hydrocarbon chain.

Preferred mineral oils include the Marcol technical range of oils (ex. Esso) although particularly preferred is the Sirius range (ex. Silkolene) or Semtol (ex. Witco Corp.). The molecular weight of the mineral oil is typically within the range 100 to 400.

One or more oils of any of the above mentioned types may be used.

It is believed that the oil provides excellent perfume delivery and also increases perfume longevity upon storage.

The oil may be present in an amount from 0.1 to 40% by weight, more preferably 0.2-20%, by weight, most preferably 0.5-15% by weight based on the total weight of the encapsulate.

(e) Short Chain Alcohols or Oils.

Preferred short chain alcohols or oils are low molecular weight, having a molecular weight of preferably 180 or less.

Monohydric or polyhydric alcohols are preferable. They typically have carbon chain length of from C1 to C9, in particular C1 to C6, and especially C1 to C4.

The presence of a lower molecular weight alcohol may help to improve physical stability upon storage by lowering the viscosity to a more desired level; it may also assists the formation of a micro-emulsion.

Examples of suitable alcohols include ethanol, isopropanol, n-propanol, dipropylene glycol, t-butyl alcohol, hexylene glycol, and glycerol.

The alcohol is preferably present in an amount from 0.1% to 40% by weight, more preferably from 0.2% to 35%, most preferably 0.5 to 20% by weight based on the total weight of the encapsulate.

(f) Electrolytes.

When employed, an electrolyte may be inorganic or organic.

Preferably the electrolyte is present in an amount from 0.001 to 1.5%, more preferably 0.01 to 1%, most preferably 0.02 to 0.7% by weight based on the total weight of the encapsulate.

Suitable inorganic electrolytes include sodium sulphate, sodium chloride, calcium(II) chloride, magnesium(II) chloride, potassium sulphate and potassium chloride.

Suitable organic electrolytes include sodium acetate, potassium acetate, sodium citrate, potassium citrate, and sodium benzoate.

The electrolyte improves viscosity control (especially viscosity reduction) of the encapsulate and assists its dispersion upon release.

The polymeric film generally comprises a polymeric backbone derived from a polymer which is water soluble.

In the context of this invention, “solubility” may be understood to refer dissolution or dispersion of a material at 20° C. and “water soluble” may be understood to mean that a material is dissolvable or dispersible at a level of 0.1 g·dm⁻³ or greater at 20° C.

The polymeric film comprises a polymeric backbone derived from a polymer which is preferably water dissolvable or dispersible at a level of 0.3 g·dm⁻³ or greater, more preferably at a level of 0.5 g·dm⁻³ or greater, at 20° C.

The polymeric film generally comprises a hydrophobically-modified polyol, in particular a hydrophobically-modified PVOH.

The polymeric film used in the invention is a material whose solubility in water is dependent upon the concentration of the surfactant present. In general, the lower the concentration of surfactant, the greater the solubility of the polymer film and the faster it breaks down.

Without wishing to be bound by theory, it is believed that hydrophobic elements within the polymeric film interact with the surfactant to form a gelled network which renders the surfactant-bound film insoluble; however, the interactions between the polymeric film and the surfactant break down on dilution and/or heating of the delivery system, thereby enabling the polymeric film to dissolve and the active agent to be released.

A preferred method according to the invention involves heating of the delivery system. Such a method is of particular benefit in the delivery of active agents, in particular cosmetic actives and pharmaceutical actives, to the human body. In such applications, the temperature increase on contact of the delivery system with the body may trigger the release of the active ingredient.

In general, the active agent is released from the polymer film encapsulate quicker when suspended in demineralised water than when suspended in an aqueous solution of the surfactant present in the delivery system. In preferred embodiments, the time taken for the release of the active agent from the polymer film is considerably less in water than in an aqueous solution of the surfactant present in the delivery system. At 20° C., the time taken in water may be less than a third, in particular, less than one seventh, of the time taken in an aqueous solution of surfactant concentration 5 g·dm⁻³.

The derivatising group referred to in the first aspect of the invention is derived from a parent material having a ClogP of from 0.5 to 6, more preferably from 1 to 6, most preferably from 2 to 6, e.g. 3 to 6.

In the context of the present invention, ClogP is calculated according to the ClogP Calculator Version 4, available from Daylight Chemicals Inc.

When a polyol is derivatised using the derivatising group, a hydrophobically-modified polyol is obtained, as referred to in the second aspect of the invention. The polyol is hydrophobically-modified by the derivatising group.

Preferred derivatising groups include those based on parent groups selected from acetals, ketals, esters, fluorinated organic compounds, ethers, alkanes, alkenes, aromatics. Especially preferred parent groups are aldehydes such as butyraldehyde, octyl aldehyde, dodecyl aldehyde, 2-ethyl hexanal, cyclohexane carboxy-aldehyde, citral, and 4-aminobutyraldehyde dimethyl acetal, although it will be readily apparent to the person skilled in the art that other suitable parent groups having the requisite ClogP are also suitable for use in the polymeric film of the invention.

Particularly preferred derivatising groups are acetals, which may derived from aldehydes or their functional equivalents (eg. dimethyl- or diethylacetals).

Preferred hydrophobically-modified polyols are hydrophobically-modified by acetal groups, in particular those having from 4 to 22 carbon atoms, and especially aromatic groups such as benzaldehyde derivatives. Hydrophobic modification using aromatic aldehydes has been found to deliver polymer films having superior interactions with surfactants, leading to better performing delivery systems. Substituted benzaldehydes, such as 2-benzaldehyde sulphonic acid and its salts may also be used.

Additional modifying groups may be present on the polymer backbone. For instance, amines may preferably be included as a modifying group since this makes the polymer more soluble in response to, for instance, the change in pH and/or ionic strength.

The derivatising group may comprise a hydrocarbyl chain. Such a hydrocarbyl chain may be optionally substituted with one or more hetero-atoms, such as oxygen or nitrogen.

The hydrocarbyl chain length of the derivatising group attached to the polymeric backbone is preferably from 3 to 22, more preferably from 4 to 18, even more preferably from 4 to 15, most preferably from 4 to 10, e.g. from 4 to 8. Hydrocarbyl chain lengths shorter than 3 are undesirable as, in use, the gel-like structure formed at the interface of the polymeric film and the surfactant will typically be too weak and will allow the polymer film to rupture too easily.

Hydrocarbyl chain lengths greater than 22 are undesirable as the parent material from which the derivatising group is obtained reacts poorly or not at all with the polymeric backbone.

The hydrocarbyl chain length of the parent material from which the derivatising group is obtained is preferably from 3 to 22, more preferably from 4 to 18.

In this context, the number of carbons in the hydrocarbyl group includes any carbon within the chain attached to any other functional group within the derivatising material.

For instance, butyraldehyde has a hydrocarbyl chain length of 4.

The derivatising material is preferably present in the polymer at a level of from 0.1 to 40% by weight, based on the total weight of the polymer, more preferably. 2 to 30%, most preferably 5 to 15%, e.g. 8 to 12%.

Where the polymeric backbone is based on PVOH, the derivatising material is preferably present at a level such that the number ratio of the derivative groups to the free hydroxyl pairs on the backbone is from 1:3 to 1:30, more preferably 1:4 to 1:20, most preferably 1:7 to 1:15, e.g. 1:8 to 1:13.

Below a ratio of 1:30, the solubility of the polymer film tends to be too great, even in the presence of surfactant. Above a ratio of 1:3, the solubility of the polymer film tends to be too low, even in the absence of surfactant.

Preferred polymers from which the backbone of the derivatised polymeric film of the invention is formed include water-soluble resins such as PVOH, cellulose ethers, polyethylene oxide (hereinafter referred to as “PEO”), starch, polyvinylpyrrolidone (hereinafter referred to as “PVP”), polyacrylamide, polyvinyl methyl ether-maleic anhydride, polymaleic anhydride, styrene maleic anhydride, hydroxyethylcellulose, methylcellulose, polyethylene glycols, carboxymethylcellulose, polyacrylic acid salts, alginates, acrylamide copolymers, guar gum, casein, ethylene-maleic anhydride resin series, polyethyleneimine, ethyl hydroxyethylcellulose, ethyl methylcellulose, hydroxyethyl methylcellulose. Water-soluble, PVOH film-forming resins are particularly preferred.

Generally, preferred water-soluble, PVOH-based film-forming polymers should have relatively low average molecular weight and high levels of hydrolysis. PVOH-based polymers preferred for use herein have an average molecular weight of from 1,000 to 300,000, preferably from 2,000 to 100,000, most preferably from 2,000 to 75,000. The level of hydrolysis is defined as the percent completion of the reaction where acetate groups on the resin are substituted with hydroxyl, —OH, groups (PVOH being derived from poly(vinyl acetate) by hydrolysis). A hydrolysis range of from 60-99% is preferred, while a more preferred range of hydrolysis is from about 88-99%. As used in this application, the term “PVOH” includes poly(vinyl acetate) compounds with levels of hydrolysis disclosed herein.

Preferred PVOH polymers have a viscosity as a 7% solution of from 100 to 5000 mPa·s at ambient temperature when measured at a shear rate of 20 s⁻¹.

All of the above polymers include the aforementioned polymer classes whether as single polymers or as copolymers formed of monomer units or as copolymers formed of monomer units derived from the specified class or as copolymers wherein those monomer units are copolymerised with one or more comonomer units.

A particularly preferred polymer/polyol for use in the present invention is represented by the formula: 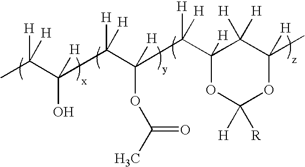
wherein the average number ratio of z to x is within the range of from 1:200 to 1:6, more preferably from 1:100 to 1:8, most preferably from 1:50 to 1:12, e.g. 1:30 to 1:14, y is the residual acetate remaining from the hydrolysis of the parent compound, which is preferably in the range of from 1-20%, more preferably 1-10%, most preferably 1-5% and R is an alkyl, alkenyl, or aryl group having from 3 to 22 carbon atoms. More preferably R is an alkyl group having from 3 to 6 carbon atoms or an aryl group.

In order to provide structural strength to the polymeric film, a degree of polymeric cross-linking is desirable. Suitable cross-linking agents include formaldehyde; polyesters; epoxides, amidoamines, anhydrides, phenols; isocyanates; vinyl esters; urethanes; polyimides; arylics; bis(methacrylkoxypropyl) tetramethylsiloxane (styrenes, methylmethacrylates); n-diazopyruvates; phenyboronic acids; cis-platin; divinylbenzene; polyamides; dialdehydes; triallyl cyanurates; N-(−2-ethanesulfonylethyl)pyridinium halides; tetraalkyltitanates; mixtures of titanates and borates or zirconates; polyvalent ions of Cr, Zr, Ti; dialdehydes, diketones; alcohol complexes of organotitanates, zircoates and borates and copper (II) complexes.

A preferred cross-linking agent is boric acid or one of its salts, e.g. sodium borate.

The level of cross-linking agent, if present, is from about 0.05% to 9% by weight of the film, more preferably from about 1% to 6%, most preferably from about 1.5% to 5% by weight. The upper range will, of course, result in more cross-linking and a slower rate of dissolution or dispersion of the film in the rinse cycle.

Functionally, it is believed that the cross-linking agent reduces the solubility of the film polymer by increasing its effective molecular weight. While it is preferred to incorporate the cross-linking agent directly into the film polymer, it is also within the scope of the invention to maintain the film in contact with the cross-linking agent during use. This may be done by adding the cross-linking agent during use or by encasing it within the film polymer. If the cross-linking agent is added in this manner, somewhat higher levels are needed to sufficiently cross-link the film polymer, and should range from about 1-15% by weight.

For PVOH-based films, the preferred cross-linking agent is a metalloid oxide such as borate, tellurate, arsenate, and precursors thereof. Other known cross-linkers include the vanadyl ion, titanium ion in the plus three valence state, or a permanganate ion (disclosed in U.S. Pat. No. 3,518,242). Alternative cross-linkers are given in the book: Polyvinylalcohol—Properties and applications, Chapter 9 by C. A. Finch (John Wiley & Sons, New York, 1973).

The polymeric film preferably incorporates a plasticiser and/or crystallinity disrupter.

It is to be understood that the term “plasticiser” and phrase “crystallinity disrupter” are interchangeable such that a reference to one is an implicit reference to the other.

The plasticiser influences the way the polymer chains react to external factors such as compression and extensional forces, temperature and mechanical shock by controlling the way that the chains distort/realign as a consequences of these intrusions and their propensity to revert or recover to their former state. The key feature of plasticisers is that they are highly compatible with the film, and are normally hydrophilic in nature.

The plasticiser will depend on the nature of the film in question.

Generally, plasticisers suitable for use with PVOH-based films have —OH groups, aiding compatibility with the —CH2-CH(OH)—CH2-CH(OH)— polymer chain of the film polymer.

Their mode of functionality is to introduce short chain hydrogen bonding with the chain hydroxyl groups and this weakens adjacent chain interactions which inhibits swelling of the aggregate polymer mass—the first stage of film dissolution.

Water itself is a suitable plasticiser for PVOH films but other common plasticisers include: polyhydroxy compounds, e.g. glycerol, trimethylolpropane, diethylene glycol, triethylene glycol, sorbitol, dipropylene glycol, polyethylene glycol; starches, e.g. starch ether, esterificated starch, oxidized starch and starches from potato, tapioca and wheat; cellulosics/carbohydrates, e.g. amylopectin, dextrin carboxymethylcelluose and pectin. Amines are a class of particularly preferred plasticisers. Dipropylene glycol may also be particularly effective.

PVP films exhibit excellent adhesion to a wide variety of surfaces, including glass, metals, and plastics. Unmodified films of polyvinylpyrrolidone are hygroscopic in character. Dry polyvinylpyrrolidone film has a density of 1.25 g·cm⁻³ and a refractive index of 1.53. Tackiness at higher humidities may be minimized by incorporating compatible, water-insensitive modifiers into the polyvinylpyrrolidone film, such as 10% of an aryl-sulfonamide-formaldehyde resin.

Suitable plasticisers for PVP-based films may be chosen from one or more of: phosphates e.g. tris(2-ethylhexyl)phosphate, isopropyl diphenyl phosphate, tributoxyethylphosphate; polyols, e.g. glycerol, sorbitol, diethylene glycol diperlargonate, polyethylene glycol di-2-ethylhexanoate, dibutyl tartrate; polyol esters, e.g. hydroxy containing polycaprolactones, hydroxy containing poly-L-lactide; lower phthalates, e.g. dimethyl phthalate, diethyl phthalate, dibutyl pthalate; and sulfonamides, e.g. toluene sulfonamide, N-ethyltoluene sulfonamide.

Preferred water-soluble films may also be prepared from PEO resins by standard moulding techniques such as calendering, casting, extrusion, and other conventional techniques. The polyethylene oxide films may be clear or opaque, and are inherently flexible, tough, and resistant to most oils and greases. These polyethylene oxide resin films provide better solubility than other water-soluble plastics without sacrificing strength or toughness. The excellent ability to lay flat, stiffness, and sealability of water-soluble polyethylene oxide films make for good machine handling characteristics.

Suitable plasticisers for PEO-based films may be selected from one or more of: phosphates, e.g. tris(2-ethylhexyl)phosphate, isopropyl diphenyl phosphate, tributoxyethylphosphate; polyols, e.g. glycerol, sorbitol, diethylene glycol diperlargonate, polyethylene glycol di-2-ethylhexanoate, dibutyl tartrate; lower phthalates, e.g. dimethyl phthalate, diethyl phthalate, dibutyl pthalate; and sulphonamides, e.g. toluene sulphonamide, N-ethyltoluene sulphonamide.

The preferred amount of plasticiser in the delivery system is from 0.001% to 25%, more preferably from 0.005% to 4% by weight.

The plasticiser and/or crystallinity disrupter may be physically bound to the backbone of the polymeric film and/or it may be present in part of the delivery system that merely comes into contact with the polymeric film. A suitable method of chemically bonding the plasticiser to the backbone of the polymeric material is described in DE 10229213.2.

A protective material which provides a barrier between the film and its contents may be present between the encapsulating polymeric film and the active agent. Such a barrier can enhance stability of the polymeric film when the active agent is present as part of an aqueous composition. A particularly suitable protective barrier material is PTFE, as disclosed in U.S. Pat. No. 4,416,791.

It is also envisaged that the polymeric film can be further protected from premature disintegration by a providing an adsorbed or coated layer of surfactant on the outside of the encapsulating polymeric film. For instance, the film may be dusted with surfactant or the film may be cast in the presence of surfactant.

Film forming on a laboratory scale can be conducted by adding an aqueous solution of the polymer, containing any plasticizers etc. to a PTFE bed, and allowing the film to form over 1 to 5 days. The resulting film thickness is nominally between 50 to 200 microns (dependent upon concentration of polymer solution, and the surface area of the PTFE bed.

The aqueous polymer solution can be cast to a controlled thickness on a commercial scale using conventional methods and techniques known in the art such as solution casting and thermo-forming techniques.

Typically, in solution casting, the aqueous polymer solutions are cast on a plate or belt using a film applicator where they are allowed to dry. The films can then be vacuum dried, air dried etc. followed by removal from the belt/plate. Casting techniques are described in U.S. Pat. No. 5,272,191 issued Dec. 21, 1993, to Ibrahim et. al. which is incorporated herein for reference.

Films can also be prepared using a melt process, which typically involves mixing the polymer with sufficient water to melt below its decomposition temperature. The blended polymer and water matrix is then fed to an extruder, extruded under tension through an appropriate die, cooled with air and taken up by an appropriate collection device. For making films, a tubular film can be made by blowing cool air through the centre of the tube to cool the film and to impart a biaxial stress to the film. Extrusion processes can also be used to make other shaped articles by using appropriate dies and moulds. Examples of such thermo forming processes are described in more detail in U.S. Pat. No. 5,646,206 issued Jul. 8, 1997, to Coffin et Al. incorporated herein by reference.

The polymeric film generally forms a “delayed release package” encapsulating the active agent. A delayed release package is one which remains intact during storage and then disperses or dissolves upon encountering conditions that reduce the concentration of surfactant adsorbed to its surface. For example, a delayed release package encapsulating a fabric conditioning agent may remain intact during storage and also during the main wash cycle of a domestic wash cycle; however, on passage into the rinse cycle, release of the encapsulate may be triggered by the reduced concentration of surfactant in the environment surrounding the polymer film, this leading to a reduced concentration of surfactant adsorbed to the surface of the polymeric film.

A trigger source in addition to absorbed surfactant depletion may also be employed. Suitable examples include those described in WO-A1-02/102956 such as sources/materials for causing changes in pH, temperature, electrolytic conditions, light, time or molecular structure. Such triggers may be used in combination with each other.

The active agent itself may also aid and/or control the dissolution or and/or dispersion of the polymeric film.

The polymeric film preferably has an average thickness of from 50 to 500 μm, more preferably from 60 to 300 μm, most preferably from 65 to 250 μm.

Typically, the delayed release package will be in the form of a pouch containing the active agent. Alternatively, or additionally, the package may comprise a network or matrix of the film and active agent, a physical and/or chemical interaction existing between the film and the active agent.

The delayed release package may be filled in a number of different ways. “Filling” refers to complete filling or partial filling whereby some air or other gas is also trapped within the delayed release package.

The delayed release package is preferably formed by horizontal or vertical form-film-seal technique.

(a) Horizontal Form-Fill-Seal

Water soluble packages based on derivatised PVOH can be made according to any of the horizontal form-fill-seal methods described in any of WO-A-00/55044, WO-A-00/55045, WO-A-00/55046, WO-A-00/55068, WO-A-00/55069 and WO-A-00/55415.

By way of example, a thermoforming process is now described where a number of delayed release packages are produced from two sheets of water soluble material. In this regard recesses are formed in the film sheet using a forming die having a plurality of cavities with dimensions corresponding generally to the dimensions of the packages to be produced. Further, a single heating plate is used for thermoforming the film for all the cavities, and in the same way a single sealing plate is described.

A first sheet of derivatised PVOH film is drawn over a forming die so that the film is placed over the plurality of forming cavities in the die. In this example each cavity is generally dome shape having a round edge, the edges of the cavities further being radiussed to remove any sharp edges which might damage the film during the forming or sealing steps of the process. Each cavity further includes a raised surrounding flange. In order to maximise package strength; the film is delivered to the forming die in a crease free form and with minimum tension. In the forming step, the film is heated to 100 to 120° C., preferably approximately 110° C., for up to 5 seconds, preferably approximately 700 micro seconds. A heating plate is used to heat the film, which plate is positioned to superpose the forming die. During this preheating step, a vacuum of 50 kPa is pulled through the pre-heating plate to ensure intimate contact between the film and the pre-heating plate, this intimate contact ensuring that the film is heated evenly and uniformly (the extent of the vacuum is dependant of the thermoforming conditions and the type of film used, however in the present context a vacuum of less than 0.6 kPa was found to be suitable). Non-uniform heating results in a formed package having weak spots. In addition to the vacuum, it is possible to blow air against the film to force it into intimate contact with the preheating plate.

The thermoformed film is moulded into the cavities blowing the film off the heating plate and/or by sucking the film into the cavities thus forming a plurality of recesses in the film which, once formed, are retained in their thermoformed orientation by the application of a vacuum through the walls of the cavities. This vacuum is maintained at least until the packages are sealed. Once the recesses are formed and held in position by the vacuum, an active agent is added to each of the recesses. A second sheet of derivatised PVOH film is then superposed on the first sheet across the filled recesses and heat-sealed thereto using a sealing plate. In this case the heat sealing plate, which is generally flat, operates at a temperature of about 140 to 160° C., and contacts the films for 1 to 2 seconds and with a force of 8 to 30 kg/cm², preferably 10 to 20 kg/cm². The raised flanges surrounding each cavity ensure that the films are sealed together along the flange to form a continuous seal. The radiussed edge of each cavity is at least partly formed by a resiliently deformable material, such as for example silicone rubber. This results in reduced force being applied at the inner edge of the sealing flange to avoid heat/pressure damage to the film.

Once sealed, the packages formed are separated from the web of sheet film using cutting means. At this stage it is possible to release the vacuum on the die, and eject the formed packages from the forming die. In this way the packages are formed, filled and sealed while nesting in the forming die. In addition they may be cut while in the forming die as well.

During the forming, filling and sealing steps of the process, the relative humidity of the atmosphere is controlled to ca. 50% humidity. This is done to maintain the heat sealing characteristics of the film. When handling thinner films, it may be necessary to reduce the relative humidity to ensure that the films have a relatively low degree of plasticisation and are therefore stiffer and easier to handle.

(b) Vertical Form-Fill-Seal

In the vertical form-fill-seal (VFFS) technique, a continuous tube of flexible plastics film is extruded. It is sealed, preferably by heat or ultrasonic sealing, at the bottom, filled with the active agent, sealed again above the active agent and then removed from the continuous tube, e.g. by cutting.

The surfactant that is used on the outside of the polymer film may be a nonionic, cationic, anionic, zwitterionic, or amphoteric surfactant.

The surfactant may be present as a coating on the surface of the polymer film or it may be present in a solution or suspension surrounding the encapsulated active agent. In embodiments in which the surfactant is present in a surrounding solution, the surfactant in solution is typically in equilibrium with surfactant absorbed to the outer surface of the encapsulating polymer film. The level of surfactant absorbed to the outer surface of the encapsulating polymer film should be sufficient to maintain the integrity of the film.

The methods of delivering an active agent according to the invention typically involve a reduction in the level of surfactant absorbed to the outer surface of the encapsulating polymer film. This reduction is generally by 25% or more, in particular by 50% or more, and especially by 75% or more of the level of surfactant absorbed to the outer surface of the encapsulating polymer film prior to reduction.

The reduction in the level of surfactant absorbed to the outer surface of the encapsulating polymer film may be brought about by dilution and/or heating of the delivery system. Dilution generally involves reducing the concentration of dissolved surfactant in a surrounding solution by 25% or more, in particular by 50% or more, and especially by 75% or more. Dilution can be suitable for triggering the release of encapsulated agro-chemicals, the dilution resulting from addition of rain water to the surfaces of the encapsulating polymeric film.

Heating of the delivery system often involves warming to body temperature or above. This can be a particularly suitable method for the delivery of pharmaceutical agents or cosmetic active agents to the human body. Heating can also be a suitable for triggering the release of encapsulated agro-chemicals, the temperature increase coming as a result of seasonal change in the weather. Heating to body temperature is an especially suitable method for delivery of cosmetic active agents to the surface of the human body. Use of heating in delivery methods according to the invention generally involves an increase in temperature of the delivery system of 5° C. or greater, in particular 10° C. or greater, and especially 15° C. or greater.

The total level of surfactant on the outside of the polymer may be from 0.001%, in particular from 0.01%, and especially from 0.1% of the weight of the polymeric film—these minimum levels being required to ensure adequate stability for the film. In order for the level of surfactant on the surface of the polymeric film to be diluted sufficiently for film rupture (when desired), it may be important for the total level of surfactant to be not too high. In general, the surfactant is present at 10% or less, in particular at 5% or less, and especially at 1% or less of the weight of the polymeric film.

Suitable nonionic surfactants include addition products of ethylene oxide and/or propylene oxide with fatty alcohols, fatty acids and fatty amines.

Preferred nonionic surfactants are substantially water soluble surfactants of the general formula:
R—Y—(C₂H₄O)_{z}—C₂H₄OH
where R is selected from the group consisting of primary, secondary and branched chain alkyl and/or acyl hydrocarbyl groups; primary, secondary and branched chain alkenyl hydrocarbyl groups; and primary, secondary and branched chain alkenyl-substituted phenolic hydrocarbyl groups; the hydrocarbyl groups having a chain length of from 8 to about 25, preferably 10 to 20, e.g. 14 to 18 carbon atoms;
and where Y is O, CO.O, or CO.N(R) in which R has the meaning given above or can be hydrogen; and Z is preferably from 8 to 40, more preferably from 10 to 30, most preferably from 11 to 25, e.g. 12 to 22.

The level of alkoxylation, Z, denotes the average number of alkoxy groups per molecule.

Preferred nonionic surfactants have an HLB of from about 7 to about 20, more preferably from 10 to 18, e.g. 12 to 16.

Typical nonionic surfactants include straight-chain and branched-chain, primary and secondary alcohol alkoxylates; alkyl phenol alkoxylates; olefinic alkoxylates; and polyol based surfactants.

Suitable straight-chain, primary alcohol alkoxylates include the deca-, undeca-, dodeca-, tetradeca-, and pentadecaethoxylates of n-hexadecanol, and n-octadecanol. Exemplary ethoxylated primary alcohols useful herein are C₁₈ EO(10); and C₁₈ EO(11). The ethoxylates of mixed natural or synthetic alcohols in the “tallow” chain length range are also useful. Specific examples of such materials include tallow alcohol-EO(11), tallow alcohol-EO(18), and tallow alcohol-EO (25), coco alcohol-EO(10), coco alcohol-EO(15), coco alcohol-EO(20) and coco alcohol-EO(25).

Suitable straight-chain, secondary alcohol alkoxylates include the deca-, undeca-, dodeca-, tetradeca-, pentadeca-, octadeca-, and nonadeca-ethoxylates of 3-hexadecanol, 2-octadecanol, 4-eicosanol, and 5-eicosanol. Exemplary ethoxylated secondary alcohols useful herein are: C₁₆ EO(11); C₂₀ EO(11); and C₁₆ EO(14).

Suitable alkyl phenol alkoxylates are the hexa- to octadeca-ethoxylates of alkylated phenols, particularly monohydric alkylphenols. The hexa- to octadeca-ethoxylates of p-tri-decylphenol, m-pentadecylphenol, and the like, are also useful herein. Exemplary ethoxylated alkylphenols useful as the viscosity and/or dispersibility modifiers of the mixtures herein are: p-tridecylphenol EO(11) and p-pentadecylphenol EO(18).

Suitable branched chain primary and secondary alcohols are available from the well-known “OXO” process.

Suitable polyol based surfactants include sucrose esters such sucrose monooleates, alkyl polyglucosides such as stearyl monoglucosides and stearyl triglucoside and alkyl polyglycerols.

Preferred cationic surfactants for use on the outside of the polymer film are the single chain cationic surfactants as described earlier as formulation and/or dispersion aids for the encapsulate. The particular options and preferences that are suitable for the formerly described purpose are also suitable for this latter purpose.

Suitable anionic surfactants for use on the outside of the polymer film include those typically used in the domestic laundry process. Examples of suitable anionic surfactants are linear alkylbenzene sulphonates, particularly linear alkylbenzene sulphonates having an alkyl chain length of C₈-C₁₅; primary and secondary alkyl sulphates, particularly C₈-C₁₅ primary alkyl sulphates; alkyl ether sulphates; olefin sulphonates; alkyl xylene sulphonates; dialkyl sulphosuccinates; and fatty acid ester sulphonates. Sodium salts are generally preferred.

Whilst perfume may be present as part of the encapsulate (vide supra), it may be present in any part of the delivery system. The following comments apply to any perfume present in the delivery system, independent of its location.

Preferred perfumes are lipophilic in nature, typically having a solubility in water of 0.01 g/ml or less, in particular 0.005 g/ml, and especially 0.003 g/ml in water at 20° C. Such perfumes may be referred to as water-insoluble perfumes.

Typical perfumes suitable for use in the present invention contain a number of ingredients which may be natural products or extracts such as essential oils, absolutes, resinoids, resins etc. and synthetic perfume components such as hydrocarbons, alcohols, aldehydes, ketones ethers, acids, esters, acetals, ketals, nitrites, phenols, etc. including saturated and unsaturated compounds, aliphatic, alicyclic, heterocyclic and aromatic compounds. Examples of such perfume components are to be found in “Perfume and Flavour Chemicals” by Steffen Arctander (Library of Congress catalogue card no. 75-91398).

The delivery systems of the invention, in particular the encapsulates, may contain one or more further components conventionally included in its particular product type; examples include pH buffering agents, perfume carriers, fluorescers, colourants, hydrotropes, antifoaming agents, antiredeposition agents, polyelectrolytes, enzymes, optical brightening agents, pearlescers, anti-shrinking agents, anti-wrinkle agents, anti-spotting agents, germicides, fungicides, anti-corrosion agents, drape imparting agents, anti-static agents, ironing aids crystal growth inhibitors, anti-oxidants, anti-reducing agents and dyes.

The invention will now be further illustrated with reference to the following non-limiting examples. All amounts are % by weight, unless otherwise stated.

Tables 1 and 2 illustrates encapsulate compositions suitable for use as fabric softener compositions. The compositions were prepared by methods known in the art. They may be encapsulated in a polymeric film and combined with a surfactant on the outside of the polymer film in accordance with the invention.

TABLE 1

| Composition | 1 | 2 |
|---|---|---|
| Quat^{a} | 93-99 | — |
| Quat^{b} | — | 22.8 |
| Sirius M85^{c} | — | 39.2 |
| ER 290^{d} | — | 15 |
| Hexylene Glycol | — | 10 |
| Tergitol 15-S-7^{e} | — | 6 |
| Perfume | 1-4 | 4 |
| Water | 0-5 | 3 |

^{a}Tetranyl AOT-1 ex Kao (80% active in 20% dipropylene glycol);
^{b}dihardened tallow dimethyl ammonium chloride (75% active in 25% propylene glycol);
^{c}branched mineral oil average molecular weight 288, ex Fuchs;
^{d}50% esterified sucrose erucate, ex Mitsubishi Foods;
^{e}Secondary alkyl alcohol with an average degree of ethoxylation of 7, ex Union Carbide.

TABLE 2

| Composition | 3 | 4 | 5 | 6 |
|---|---|---|---|---|
| Quat^{a} | 35 | 35 | 35 | 35 |
| Perfume | 3 | 3 | 3 | 3 |
| Estol 1545^{b} | 27 | 27 | 27 | 27 |
| Estasol^{c} | 10 | | | |
| NMP^{d} | | 10 | | |
| DMSO^{e} | | | 10 | |
| Benzyl alcohol | | | | 10 |
| Coco-3^{f} | 5 | 5 | 5 | 5 |

^{a}1,2-ditallowoyloxy ethyl,3-trimethyl ammoniopropane chloride
^{b}ester oil
^{c}mixture of methyl esters of adipic, glutaric and succinic acids
^{d}N-methyl pyrrolidone
^{e}Dimethyl sulphoxide
^{f}Coco-alcohol 3 EO

Further encapsulates suitable for use as fabric softener compositions may be prepared in the following manner.

A substantially non-aqueous melt can be prepared by heating a reaction vessel to at least 50° C., adding an oil and a nonionic surfactant to the vessel and stirring the mixture. A cationic surfactant and a fatty acid and/or a long or short chain alcohol are then added to the vessel, and the stirring rate is increased. Stirring is continued until a homogenous mixture is formed. The mixture is then left to cool to ambient temperature, under continuous stirring. Optionally perfume and/or a polymeric structurant (such as disclosed in WO99/43777) is then stirred into the mixture.

A substantially non-aqueous microemulsion is prepared by mixing under low agitation an oil, a solvent such as a low molecular weight alcohol, a dispersibility aid such as a nonionic surfactant, a cationic surfactant and 10% by weight or less of water until a clear composition is formed. In order to assist formation of the clear microemulsion, the mixture may be heated as required. Perfume may optionally be added to the mixture at any stage.

A substantially non-aqueous concentrated emulsion is prepared by heating water to a temperature above 50° C., adding an emulsifier, premixing a cationic surfactant, nonionic surfactant and oil and adding this to the water. Optionally the product is milled and then allowed to cool. Once below 50° C., perfume may be added.

Preparation of Polymeric Materials

A 10 wt % solution of PVOH in water was prepared by placing 10 g PVOH (Mowiol 20-98 (trade name), ex Kuraray Specialities) and 900 g demineralised water into a flask and heating to 70° C. To this, 10 ml of hydrochloric acid (36% aqueous solution) was added to catalyse the reaction and then butyraldehyde was added. The mixture was then stirred at 70° C. for 5 hours under an inert atmosphere, after which time the heating was stopped and agitation continued for a further 20 hours at room temperature. The reaction mixture was then brought to a pH of 7 using a sodium hydroxide solution.

The resulting solution was precipitated into acetone to yield the acetalised PVOH polymer and washed repeatedly with acetone (500 ml) and then water (50 ml). It was then dried under vacuum at 70° C. overnight to yield a white polymer.

The extent of acetalisation was analysed to be 10.4%.

Preparation of Polymeric Film

The poly(vinyl alcohol)-butyral (PVA-BA) resin prepared above was diluted to a 7% m/m. solution with demineralized water. The resulting solution was poured onto a PTFE glued-sheet tray. The polymer solution was then left to evaporate to produce films. The thickness of the films was adjusted by increasing or decreasing the volume of liquid polymer dosed in a given space. After 2 to 3 days, the films were peeled away from the PTFE tray, and an average thickness was measured at 5 regions of the cast films using an electronic micrometer. The films were then stored at 23° C. and 50% relative humidity for 2 days prior to evaluation.

The following examples illustrate the effect of anionic/nonionic surfactant concentration on the butyraldehyde-derivatised PVOH. The slide-test method described below was employed as a screen for the polymer films.

Film Rupture Testing

The evaluation of the effect of anionic/nonionic surfactant concentration on the polymer material is made based on its dissolution and erosion characteristics using a slide-testing regime.

This is denoted by the rupture time, i.e. the first time when the polymer breaks and the contents flow from the inside of the sachet into the surrounding liquid.

A film slide was used to hold a 30 mm×30 mm film cast to a thickness of 100-200 μm, in place. The slide and film were then immersed in either a detergent surfactant solution or tap water in a 1 litre beaker. The slide and film to be tested were stirred at ambient temperature at 293 rpm until the polymer film ruptured.

Films were prepared from the polymer synthesised above and the nature of the films tested is given in Table. The results of the rupture test are given in Table 4.

TABLE 3

| Sample | Film thickness^{a} | Base^{b} | Degree modified^{c} | Solids^{d} | mPa · s^{e} |
|---|---|---|---|---|---|
| 1 | 184 | 20-98 | 9 | 15.53 | 20.6 |
| 2 | 150 | 20-98 | 11 | 15.6 | 20.8 |
| 3 | Not measured | 20-98 | 12 | 15.7 | 21.1 |
| 4 | 192 | 26-88 | 10 | 15.46 | 23.4 |
| 5 | 173 | 26-88 | 12 | 15.6 | 26.2 |
| 6 | 149 | 28-99 | 10 | 10.83 | 24.2 |
| 7 | 166 | 28-99 | 11 | 10.75 | 25.6 |
| 8 | 110 | 28-99 | 12 | 10.81 | 24.11 |
| 9 | 185 | 20-98 | 10 | 15.6 | 20.7 |

^{a}μm. Average of 5 readings across the films surface;
^{b}Base hydrolyzed PVOH employed during the derivatisation (Mowiol range, ex Kuraray);
^{c}Degree of butyral modification (percentage of butyral group based on —OH pairs in the resin);
^{d}polymer content of base resin as supplied;
^{e}Viscosity at 4% m/m measured at 20° C. on a Haake Rotoviscometer at 106⁻¹ using an NV cup and bob.

TABLE 4

| | | | Rupture | Rupture | |
|---|---|---|---|---|---|
| | Cloud | Precipitation | time in | time in | |
| Sample | point^{a} | point^{b} | Detergent^{c} | water^{d} | T_{W}/T_{T}^{e} |
| 1 | <25 | 46 | 29 | 20 | 1.5 |
| 2 | <25 | 37 | 36 | 6.5 | 5.5 |
| 3 | <25 | 35 | — | — | — |
| 4 | <25 | 31 | 7 | 5 | 1.4 |
| 5 | <25 | 28 | 0.25 | 4 | 0.07 |
| 6 | 34 | 40 | 25 | 15 | 1.7 |
| 7 | 32 | 38 | 20.3 | 2.8 | 7.25 |
| 8 | 29 | 34 | 13 | 10 | 1.3 |
| 9 | <25 | 42 | 60 | 7 | 8.57 |

^{a}Temperature (° C.) at which polymer starts to become more hydrophobic due to an LCST effect;
^{b}Temperature (° C.) at which precipitation of the polymer occurs due to hydrophobic LCST behaviour;
^{c}Time (minutes) for the film to rupture in 1.66 g/L Ultra Wisk (trade name) at ambient temperature;
^{d}Time (minutes) for the film to rupture in tap-water at ambient temperature;
^{e}Ratio of rupture time in Ultra Wisk compared to tap-water.

The polymer of sample 9 was cast to a thickness of 200 μm and placed onto a slide. The effect of altering the concentration of a premium washing detergent (Ultra-Wisk, trade name) was then measured using the slide test regime at ambient temperature, as described above.

The results are given in Table 5 and clearly show that the rupture time varies significantly with level of surfactant.

TABLE 5

| Detergent^{a} g/L | Rupture Time, minutes |
|---|---|
| 0 | 7 |
| 0.008 | 13 |
| 0.016 | 18 |
| 0.035 | 29 |
| 1.66 | 65 |

^{a}Ultra-Wisk purchased in the U.S., February 2001.

A sample of polymer 9 was cast to 90 μm from a 15% solution. The resulting film was conditioned at 20° C. and 65% R.H. for 24 hours. A Tergometer was filled with 1 litre of cold Wirral water (15-20° FH) optionally containing 2 g/litre of Wisk solution (Wisk purchased from the U.S. May 2003) and set to agitate at 75 r.p.m. Immediately after agitation was started the film was placed in the pot, and visually inspected for fragmentation (inspection was stopped after 15 minutes). The test was repeated 3 times. The results are given in Table 6:

TABLE 6

| | Film | | Time to fragment |
|---|---|---|---|
| Sample | weight (g) | Solution | (minutes) |
| 1 | 0.47 | A | >15 |
| 2 | 0.38 | A | >15 |
| 3 | 0.45 | A | >15 |
| 4 | 0.39 | B | 3 |
| 5 | 0.42 | B | 7 |
| 6 | 0.53 | B | 4 |

“A” is a solution of 2 g/litre of Wisk in 1 litre of cold Wirral water
“B” is 1 litre of cold Wirral water

Fragmentation occurs when the polymeric film breaks into more than one piece.

Evaluation of Extent of Butyral Derivatisation

Films were cast using the polymer of sample 9 and various levels of butyral derivatising group (prepared as described above). The slide test method was used to measure the rupture time in detergent (T_{w}) and the rupture time in water (T_{T}).

The results are given in Table 7 and illustrate that a degree of modification above 6% of butyral significantly increases rupture time.

TABLE 7

| % Butyral | T_{W} Minutes | T_{T} Minutes | T_{W}/T_{T} |
|---|---|---|---|
| 6 | 20 | 6 | 3.33 |
| 9.3 | 40 | 16 | 2.5 |
| 12.5 | 45 | 13 | 3.46 |

T_{W} = Time for film rupture in 1.66 g/L Wisk solution
T_{T} = Time for film rupture in tap-water
T_{w}/T_{T} = Ratio of rupture time in Wisk solution:rupture time in tap-water.

Viscosity Evaluation

The sample 9 polymer was diluted to 7% using either demineralized water or 20 g/litre SDS. The viscosity of the diluted resin was then measured.

The results are given in the following Table 8 and demonstrate that the anionic surfactant is interacting with the polymeric film to create a gel-like structure.

TABLE 8

| SDS g/L | Viscosity, mPa · s^{a} |
|---|---|
| 0 | 230 |
| 20 | 970 |

^{a}Measured on a Haake Rotoviscometer at 25.4° C. and 20 s⁻¹ using an NV cup and bob.

Other Polymer Films

Numerous other polymer films were prepared by the method described. Tables 9-11 indicate the nature of the polymers prepared and their interactions with surfactants. It is clear from these results that a wide variety of polymer films have interactions with cationic, anionic and nonionic surfactants, indicating many possible embodiments of the invention.

The following protocol is a modified version of that found in European Patent, EP 0283180 by Aicello Chemical Co:

A 10% by weight homogeneous solution of the polyvinyl alcohol starting material was accurately measured into the glass reactor vessel (20 g Kuraray Mowiol 20-98 in 180 mL deionised water). The solution was warmed to 70° C. with constant stirring at ˜600 rpm (Since the reaction is very viscous it is necessary to use either an overhead motorised impeller blade or to use a heavy duty magnetic stirrer plate, use whatever gives efficient stirring). Thereafter HCl catalyst (2 mL of a 37 weight % solution in water) was added to the stirred solution. This was calculated so that the molar ratio: [HCl]/[OH polymer repeats] ˜0.056.

A dilute stock solution of aldehyde in aqueous solution is prepared and the required amount of this is added dropwise, slowly, to the reactor over ˜1 hour. After the addition is complete then the reaction is stirred for 5 hours at 70° C.

The level of derizatization achieved is indicated in the Tables.

TABLE 9

| | | Strength of |
|---|---|---|
| | | interaction |
| | | with LAS |
| Starting polymer^{a} | Derivatizing group^{b} | (anionic)^{c} |
| Mowiol 6-98 | 4 amino butyraldehyde | ++ |
| | dimethyl acetal and | |
| | 4 amino butyraldehyde | |
| | dimethyl acetal, methyl | |
| | iodide salt 50:50 (10.33% | |
| | substituted) | |
| Mowiol 6-98 | 4 amino butyraldehyde | + |
| | dimethyl acetal and | |
| | 4 amino butyraldehyde | |
| | dimethyl acetal, methyl | |
| | iodide salt 50:50 (7.58% | |
| | substituted) | |
| Mowiol 6-98 | Butyraldehyde (5.02%) and | + |
| | 4-amino butyraldehyde | |
| | dimethyl acetal, methyl | |
| | iodide salt (4.14%) | |
| Mowiol 6-98 | Butyraldehyde (3.58%) and | + (note NI |
| | 4 amino butyraldehyde | interaction |
| | dimethyl acetal, (3.52%) | also) |
| Mowiol 6-98 | Benzaldehyde (13%) | + (note CTAC |
| | | and NI |
| | | interaction) |
| Mowiol 6-98 | Benzaldehyde (6.56%) | + (note CTAC |
| | | and NI |
| | | interaction) |
| Mowiol 6-98 | 4-amino butyraldehyde | ++ |
| | dimethyl acetal methyl | |
| | iodide salt (7.06%) | |
| Mowiol 6-98 | 4-amino butyraldehyde | ++ (note NI |
| | dimethyl acetal (7.05%) | interaction) |
| Mowiol 6-98 | 2-ethyl hexanal (13%) | + (note CTAC |
| | | and NI |
| | | interaction) |
| Mowiol 10-98 | Butyraldehyde and | +++ |
| | 4-amino butyraldehyde | |
| | dimethyl acetal, methyl | |
| | iodide salt 50:50 (12.41) | |
| Mowiol 10-98 | Butyraldehyde (3.10) and | + |
| | 4-amino butyraldehyde | |
| | dimethyl acetal, methyl | |
| | iodide salt (2.16) | |
| Mowiol 10-98 | Butyraldehyde and | + (note CTAC |
| | Propionaldehyde 50:50 | and NI |
| | (9.2) | interaction) |
| Mowiol 10-98 | Butyraldehyde and | + (note CTAC |
| | Propionaldehyde 50:50 | and NI |
| | (12.9) | interaction) |
| Mowiol 10-98 | Butyraldehyde and | + (note NI |
| | Propionaldehyde 50:50 | interaction) |
| | (9.4) | |
| Mowiol 10-98 | 4-amino butyraldehyde | +++ |
| | dimethyl acetal (7.97) | |
| Mowiol 10-98 | 4-amino butyraldehyde | +++ |
| | dimethyl acetal (11.8) | |
| Mowiol 10-98 | Butyraldehyde (6.96) | + |
| Mowiol 8-88 | 4-amino butyraldehyde | ++ |
| | dimethyl acetal (6.14) | |
| Mowiol 5-88 | Butyraldehyde (6.35) and | ++ |
| | 4-amino butyraldehyde | |
| | dimethyl acetal (6.32) | |
| Mowiol 5-88 | 4-amino butyraldehyde | +++ |
| | dimethyl acetal (12.98) | |
| Mowiol 5-88 | 2-ethyl hexanal (10.02) | + (note CTAC |
| | | and NI |
| | | interaction) |
| Mowiol 20-98 | Butyraldehyde and 2-ethyl | + (note CTAC |
| | hexanal 50:50 (6.75) | and NI |
| | | interaction) |
| Mowiol 20-98 | Butyraldehyde and | + (note CTAC |
| | propionaldehyde 50:50 | and NI |
| | (13.26) | interaction) |
| Mowiol 20-98 | Butyraldehyde and | + (note CTAC |
| | propionaldehyde 50:50 | and NI |
| | (6.51) | interaction) |
| Mowiol 20-98 | Butyraldehyde (6.28) and | +++ |
| | 4-amino butyraldehyde | |
| | dimethyl acetal (6.79) | |
| Mowiol 20-98 | Benzaldehyde (12.76) | ++ (note CTAC |
| | | and NI |
| | | interaction) |
| Mowiol 20-98 | Benzaldehyde (9.67) | + (note CTAC |
| | | and NI |
| | | interaction) |
| Mowiol 20-98 | 4-amino butyraldehyde | +++ (note |
| | dimethyl acetal (10.02) | CTAC and NI |
| | | interaction) |
| Mowiol 20-98 | 2-ethylhexanal (9.19) | + (note CTAC |
| | | and NI |
| | | interaction) |
| Mowiol 20-98 | Butyraldehyde (7.10) | + |
| Mowiol (18-88) | Butyraldehyde (4.84) and | ++ |
| | 4-amino butyraldehyde | |
| | dimethyl acetal (4.26) | |
| Mowiol (18-88) | Butyraldehyde (3.31) and | + |
| | 4-amino butyraldehyde | |
| | dimethyl acetal (3.01) | |
| Mowiol 18-88 | Benzaldehyde (6.95) | + |
| Mowiol 18-88 | 4-amino butyraldehyde | +++ |
| | dimethyl acetal (13) | |
| Mowiol 18-88 | 2-ethylhexanal (12.96) | + (note CTAC |
| | | and NI |
| | | interaction) |

^{a}Ex KSE first digit relates to molecular weight based on viscosity of a 4% m/m. solution;
^{b}the material used to derivatize the PVOH (all reactions were conducted in aqueous media);
^{c}+++ = large interaction leading to precipitation; ++ = dense clouding and sometimes precipitation; + = clouding.

TABLE 10

| | | Strength of |
|---|---|---|
| | | interaction |
| | | with CTAC |
| Starting polymer^{a} | Derivatizing group^{b} | (cationic)^{c} |
| Mowiol 6-98 | Butyraldehyde (6.2) and 2- | +++ |
| | benzaldehyde sulphonic | |
| | acid sodium salt (6.52) | |
| Mowiol 6-98 | Benzaldehyde (13) | + (note LAS |
| | | and NI |
| | | interaction) |
| Mowiol 6-98 | Benzaldehyde (6.56) | + (note LAS |
| | | and NI |
| | | interaction) |
| Mowiol 6-98 | 2-benzaldehyde sulphonic | +++ |
| | acid sodium salt (9.98) | |
| Mowiol 6-98 | 2-ethylhexanal (13) | + (note LAS |
| | | and NI |
| | | interaction) |
| Mowiol 10-98 | Butyraldehyde (3.71) and | ++ (note |
| | 2-benzaldehyde sulphonic | interaction |
| | acid sodium salt (3.47) | with NI) |
| Mowiol 10-98 | Butyraldehyde and | + (note LAS |
| | propionaldehyde 50:50 | and NI |
| | (9.2) | interaction) |
| Mowiol 10-98 | Butyraldehyde and | + (note LAS |
| | propionaldehyde 50:50 | and NI |
| | (12.9) | interaction) |
| Mowiol 10-98 | Benzaldehyde (6.44) | + (note |
| | | interaction |
| | | with NI) |
| Mowiol 10-98 | 2-benzaldehyde sulphonic | +++ |
| | acid sodium salt (6.78) | |
| Mowiol 5-88 | Butyraldehyde (3.55) and | ++ |
| | 2-benzaldehyde sulphonic | |
| | acid sodium salt (3.73) | |
| Mowiol 5-88 | Benzaldehyde (9.51) | + (note |
| | | interaction |
| | | with NI) |
| Mowiol 5-88 | 2-benzaldehyde sulphonic | +++ |
| | acid sodium salt (12.92) | |
| Mowiol 20-98 | Butyraldehyde (6.63) and | + (note |
| | benzaldehyde (6.70) | interaction |
| | | with NI) |
| Mowiol 20-98 | Butyraldehyde (4.61) and | +++ |
| | 2-benzaldehyde sulphonic | |
| | acid sodium salt (5.30) | |
| Mowiol 20-98 | Butyraldehyde and 2- | + (note |
| | ethylhexanal 50:50 (6.75) | interaction |
| | | with LAS and |
| | | NI) |
| Mowiol 20-98 | Butyraldehyde and | + (note |
| | propionaldehyde 50:50 | interaction |
| | (13.26) | with LAS and |
| | | NI) |
| Mowiol 20-98 | Butyraldehyde and | + (note |
| | propionaldehyde 50:50 | interaction |
| | (6.51) | with LAS and |
| | | NI) |
| Mowiol 20-98 | Benzaldehyde (12.76) | ++ (note |
| | | interaction |
| | | with LAS and |
| | | NI) |
| Mowiol 20-98 | Benzaldehyde (9.67) | + (note |
| | | interaction |
| | | with LAS and |
| | | NI) |
| Mowiol 20-98 | 2-benzaldehyde sulphonic | +++ |
| | acid sodium salt (13.28) | |
| Mowiol 20-98 | 4-amino butyraldehyde | + (note |
| | dimethyl acetal (10.02) | interaction |
| | | with LAS and |
| | | NI) |
| Mowiol 20-98 | 2-ethylhexanal (9.19) | ++ (note |
| | | interaction |
| | | with LAS and |
| | | NI) |
| Mowiol 18-88 | Butyraldehyde (3.54) and | +++ |
| | 2-benzaldehyde sulphonic | |
| | acid sodium salt (3.57) | |
| Mowiol 18-88 | Benzaldehyde (12.84) | + (note |
| | | interaction |
| | | with NI) |
| Mowiol 18-88 | 2-benzaldehyde sulphonic | +++ |
| | acid sodium salt (9.81) | |
| Mowiol 18-88 | 2-ethylhexanal (12.96) | + (note |
| | | interaction |
| | | with NI) |

^{a,b,c}as for Table 9.

TABLE 11

| | | Strength of |
|---|---|---|
| | | interaction |
| | | (3 EO non- |
| Starting polymer^{a} | Derivatizing group^{b} | ionic)^{c} |
| Mowiol 6-98 | Butyraldehyde (3.58) and | + (note |
| | 4-amino butyraldehyde | interaction |
| | dimethyl acetal (3.52) | with LAS) |
| Mowiol 6-98 | Butyraldehyde and | + |
| | propionaldehyde 50:50 | |
| | (6.7) | |
| Mowiol 6-98 | Benzaldehyde (13) | ++ (note |
| | | interaction |
| | | with LAS and |
| | | CTAC) |
| Mowiol 6-98 | Benzaldehyde (6.56) | ++ (note) |
| | | interaction |
| | | with LAS and |
| | | CTAC) |
| Mowiol 6-98 | 4-amino butyraldehyde | + (note |
| | dimethyl acetal (7.05) | interaction |
| | | with LAS) |
| Mowiol 6-98 | 2-ethylhexanal (13) | + (note |
| | | interaction |
| | | with CTAC and |
| | | LAS) |
| Mowiol 10-98 | Butyraldehyde (3.71) and | + (note CTAC |
| | 2-benzaldehyde sulphonic | interaction) |
| | acid sodium salt (3.47) | |
| Mowiol 10-98 | Butyraldehyde and | + (note |
| | propionaldehyde 50:50 | interaction |
| | (9.2) | with LAS and |
| | | CTAC) |
| Mowiol 10-98 | Butyraldehyde and | + |
| | propionaldehyde 50:50 | |
| | (12.9) | |
| Mowiol 10-98 | Butyraldehyde and | + |
| | propionaldehyde 50:50 | |
| | (12.3) | |
| Mowiol 10-98 | Butyraldehyde and | + |
| | propionaldehyde 50:50 | |
| | (9.4) | |
| Mowiol 10-98 | Benzaldehyde (6.44) | ++ (note |
| | | interaction |
| | | with LAS and |
| | | CTAC) |
| Mowiol 10-98 | 4-amino butyraldehyde | + (note |
| | dimethyl acetal (11.8) | interaction |
| | | with LAS) |
| Mowiol 10-98 | 2-ethylhexanal (7.16) | + (note |
| | | interaction |
| | | with CTAC) |
| Mowiol 10-98 | Propionaldehyde (13.7) | + |
| Mowiol 10-98 | Butyraldehyde (6.96) | + (note LAS |
| | | and CTAC |
| | | interaction) |
| Mowiol 8-88 | Propionaldehyde (10) | + |
| Mowiol 8-88 | Propionaldehyde (6.76) | + |
| Mowiol 8-88 | Propionaldehyde (9.88) | + |
| Mowiol 5-88 | Benzaldehyde (9.51) | + (note CTAC |
| | | interaction) |
| Mowiol 5-88 | 2-ethylhexanal (10.02) | + (note |
| | | interaction |
| | | with LAS and |
| | | CTAC) |
| Mowiol 20-98 | Butyraldehyde (6.63) and | + (note CTAC |
| | benzaldehyde (6.7) | interaction) |
| Mowiol 20-98 | Butyraldehyde and 2- | ++ (note LAS |
| | ethylhexanal 50:50 (6.75) | and CTAC |
| | | interaction) |
| Mowiol 20-98 | Butyraldehyde and | + (note LAS |
| | propionaldehyde 50:50 | and CTAC |
| | (13.26) | interaction) |
| Mowiol 20-98 | Butyraldehyde and | + (note LAS |
| | propionaldehyde 50:50 | and CTAC |
| | (6.51) | interaction) |
| Mowiol 20-98 | Benzaldehyde (12.76) | +++ (note LAS |
| | | and CTAC |
| | | interaction) |
| Mowiol 20-98 | Benzaldehyde (9.67) | ++ (note LAS |
| | | and CTAC |
| | | interaction) |
| Mowiol 20-98 | 4-amino butyraldehyde | + (note LAS |
| | dimethyl acetal (10.02) | and CTAC |
| | | interaction) |
| Mowiol 20-98 | 2-ethylhexanal (9.19) | ++ (note LAS |
| | | and CTAC |
| | | interaction) |
| Mowiol 18-88 | Butyraldehyde and 2- | ++ |
| | ethylhexanal 50:50 (9.41) | |
| Mowiol 18-88 | Benzaldehyde (6.95) | + (note |
| | | interaction |
| | | with LAS and |
| | | CTAC) |
| Mowiol 18-88 | Benzaldehyde (12.84) | ++ (note |
| | | interaction |
| | | with CTAC) |
| Mowiol 18-88 | 2-ethylhexanal (12.96) | ++ (note |
| | | interaction |
| | | with LAS and |
| | | CTAC) |

^{a,b,c}as for Table 9

Temperature Trigger

The following study investigated the binding of LAS with the butyral derivatized 20-98 using isothermal calorimetry to measure the extent of the interaction. Table 12 illustrates the reduction of interaction found at increased temperatures.

TABLE 12

| Temperature (° C.) | LAS (mM) Bound to Polymer |
|---|---|
| 14 | 6.14 |
| 30 | 6.8 |
| 45 | 3.67 |
| 60 | 1.35 |

Evaluation of Film in Laundry Operation
Capsule Preparation

The sample 9 polymer was cast to form a film measuring 10 cm×10 cm and a thickness of 50 μm, 90 μm or 100 μm. This was folded in half and 3 of the 4 sides were heat sealed at 150° C. using a Hulme-Hunter heat sealer to form a pouch. 20 g of a formulation consisting of 96 wt % Tetranyl AOT-1 (a quaternary ammonium softening material based on triethanolamine, 80% active ex Kao) and 4 wt % perfume (hereinafter referred to as formulation “A”) or 20 g of a formulation comprising 96 wt % Tetranyl AOT-1, 3 wt % water and 1 wt % perfume (hereinafter referred to as formulation “B”) was then introduced into the pouch, and the top of the film sealed to form a capsule. The capsule was then stored at 23° C. and 50% relative humidity for 2 days prior to evaluation.

Machine Wash Evaluation

A top-loading washing machine (Whirlpool) was filled with 65 litres of water (60 French Hardness at 15° C.). 110 g washing liquid (Ultra Wisk) was added and gently agitated for 10 minutes until dissolved. 3.5 kg of a mixed ballast load comprising 1 kg Terry towel, 1 kg cotton poplin, 1 kg poly-cotton and 0.5 kg polyester was then added, together with ten 20 cm×20 cm Terry towel monitors, followed by the capsule formed from a 100 μm thick film containing formulation “A”. The machine was then set for an 18 minute wash at 15° C., a spin, and one rinse (5 minutes). After the wash phase the integrity of the capsule was assessed visually, and found to be very flaccid but still intact. After the programme was finished, the cloth and drum were inspected for any residual gelled polymer film. No residual film was found.

Softness Evaluation

The Terry towel monitors were retrieved and softening was assessed after tumble drying against the tumble-dried controls by a trained panel of 10 people using paired comparison testing. Results were analysed at the 95% C.I. level and are given in the Table 13.

TABLE 13

| Treatment | % Preference |
|---|---|
| Detergent only | 22 |
| Detergent & capsule | 78 |

The results clearly indicate that softening benefits were perceivable when the capsule was present.

Perfume Evaluation

The Terry towelling was also assessed by the panel (paired comparison test) for perfume preference both on damp cloth (5 hrs line dried) and after tumble drying.

The results are given in the following Table 14.

TABLE 14

| Treatment | % Preference |
|---|---|
| Detergent only - assessment before | 21 |
| tumble drying | |
| Detergent & capsule - assessment before | 79 |
| tumble drying | |
| Detergent only - assessment after | 20 |
| tumble drying | |
| Detergent & capsule - assessment after | 80 |
| tumble drying | |

The results clearly indicate that significant improvements in perfume benefits are achieved when the capsule is present in the laundry treatment process.

The investigation for gelled residue was conducted on a further 3 occasions, under the machine washing conditions described in the example above. On all three occasions no residue was found either on the cloth, drum or agitator spindle.

Further Evaluation in Laundry Operation

A Whirlpool U.S. top-loader was filled with 2.5 Kg of mixed ballast (Terry towel, poly-cotton, poly-ester, cotton sheeting) with 6 terry towel monitors (20 cm×20 cm). The machine was allowed to fill with 65 litres of cold water at 15° C., and 6° F.H. 110 g of ultra-Wisk was added. A 10 or 18 minute super-wash was selected followed by a single rinse and spin. The capsules comprising formulation “B” and unencapsulated fabric treatment compositions were added at various stages of the laundry cycle. After the cycle was complete the ballast, and the monitors were dried in a Whirlpool U.S. dryer. The monitors were then isolated, and treated with bromophenol blue stain in order to indicate the intensity and evenness of cationic softener coverage.

The bromophenol blue test consisted of bromophenol blue dye (0.7 g) dissolved in ethanol (10 g), added to hot water (5 ml) and then added to 10 litres of cold Wirral water (final pH 7.4).

The monitors were added to the bromophenol blue solution, left at ambient temperature for 15 minutes with occasional agitation and then rinsed gently until the rinse waters were clear. The clothes were then spun for 30 seconds to remove any excess water, and left to line dry away from direct sunlight.

The monitors were then visually assessed via a trained panel of 8 people for evenness of deposition on a scale of 1-5 where 1 denotes very patchy and 5 denotes complete coverage, and intensity of blue stain also on a scale of 1-5 where 1 denotes very pale and 5 denotes very dark.

In Table 15, the capsule was formed from a film cast to 50 microns and the 18 minute wash cycle was used.

TABLE 15

| Treatment | Evenness | Intensity |
|---|---|---|
| Capsule containing 20 g formulation | 3 | 4 |
| “B” added at start of wash cycle | | |
| 20 g formulation “B” added at start | 4 | 4 |
| of rinse cycle | | |
| 20 g formulation “B” added at start | 1 | 1 |
| of wash cycle | | |
| 30 ml Ultra-Snuggle added at start of | 5 | 4 |
| rinse cycle | | |
| Capsule containing 20 g formulation | 1 | 1 |
| “B” ruptured by hand and added at | | |
| start of wash cycle | | |
| 20 g formulation “B” pre-dispersed in | 5 | 4 |
| 200 ml of demineralised water and | | |
| added at start of rinse cycle | | |

In the following table, the capsule was formed from a film cast to 90 microns and the both the 10 and 18 minute wash cycles were used.

Softening was assessed by a trained panel of 6 people on a line scale of 0 to 100 where 0 denotes not at all soft and 100 denotes extremely soft. The results were analysed using Anova and Tukey-Kramer HSD statistics. Perfume was assessed by a trained panel of 8 people on a scale of 0 to 5 where 0 denotes no perfume and 5 denotes very intense perfume. Perfume assessment was made on the wet fabrics immediately after removal from the washing machine and also 24 hours after removal from the tumble dryer. The results are shown in Table 16.

TABLE 16

| | | Perfume | Perfume |
|---|---|---|---|
| Treatment | Softening | (wet) | (24 Hrs) |
| 30 ml Ultra-Snuggle | 59.2 | 2.25 | 1.88 |
| added to start of rinse | | | |
| cycle after end of 18 | | | |
| minute wash cycle | | | |
| Capsule containing 20 g | 64.1 | 2.33 | 1.98 |
| formulation “B” added | | | |
| at start of 18 minute | | | |
| wash cycle | | | |
| Capsule containing 20 g | 45.3 | 2.24 | 1.67 |
| formulation “B” added | | | |
| to start of rinse cycle | | | |
| after end of 18 minute | | | |
| wash cycle | | | |

1. A delivery system comprising an active agent encapsulated by a polymeric film comprising a polymeric backbone derived from a polymer which is water soluble, and one or more derivatising groups attached to the backbone, the derivatising group(s) being derived from a parent material having a ClogP of from 0.5 to 6, the delivery system also comprising a surfactant on the outside of the polymeric film.

2. A delivery system according to claim 1, wherein the derivatising group(s) are derived from a parent material comprising a C3 to C22 hydrocarbyl chain.

3. A delivery system according to claim 1, wherein the derivatising group(s) are derived from a parent material selected from the group consisting of acetals, ketals, esters, fluoro-organics, ethers, epoxides, alkanes, alkenes and aromatic compounds.

4. A delivery system comprising an active agent encapsulated by a polymeric film comprising a hydrophobically-modified polyol, the delivery system also comprising a surfactant on the outside of the polymeric film.

5. A delivery system according to claim 4, wherein the hydrophobically-modified polyol is a hydrophobically-modified poly(vinyl alcohol).

6. A delivery system according to claim 4, wherein the polyol is hydrophobically-modified by groups having from 3 to 22 carbon atoms.

7. A delivery system according to claim 4, wherein the polyol is hydrophobically-modified by acetal groups.

8. A delivery system according to claim 7, wherein the polyol is hydrophobically-modified by aromatic acetal groups.

9. A delivery system according to claim 8, wherein the aromatic acetal groups are derived from benzaldehyde.

10. A delivery system according to claim 7, wherein acetal groups are derived from butyraldehyde.

11. A delivery system according to claim 4, wherein the polyol comprises a number of esterified hydroxy groups on the polyol from 1 to 30%.

12. A delivery system according to claim 11, wherein the number of esterified hydroxy groups on the polyol is from 1 to 20%.

13. A delivery system according to claim 4, wherein the degree of hydrophobic modification of the polyol is from 0.1 to 40% based on the total weight of the polymer.

14. A delivery system according to claim 4, wherein the polyol comprises hydrophobic groups and pairs of free hydroxyl groups in a ratio of from 1:3 to 1:30.

15. A delivery system according to claim 1, wherein the surfactant is adsorbed onto the outside surface of the polymeric film.

16. A delivery system according to claim 1, comprising a surfactant-containing aqueous carrier fluid.

17. A delivery system according to claim 16, wherein the carrier comprises a nonionic or cationic surfactant.

18. A delivery system according to claim 1, wherein the polymeric film comprises a crystallinity disrupter and/or a plasticizer.

19. A delivery system according to claim 18, wherein the crystallinity disruptor and/or a plasticizer is di(propylene glycol).

20. A delivery system according to claim 4, wherein the surfactant is adsorbed onto the outside surface of the polymeric film.

21. A delivery system according to claim 4, comprising a surfactant-containing aqueous carrier fluid.

22. A delivery system according to claim 21, wherein the carrier comprises a nonionic or cationic surfactant.

23. A delivery system according to claim 4, wherein the polymeric film comprises a crystallinity disruptor and/or a plasticizer.

24. A delivery system according to claim 23, wherein the crystallinity disrupter and/or a plasticizer is di(propylene glycol).

25. A method of delivering an active agent to a target comprising providing a polymeric backbone derived from a polymer which is water soluble, and one or more derivatising groups attached to the backbone, the derivatising group(s) being derived from a parent material having a ClogP of from 0.5 to 6, the delivery system also comprising a surfactant on the outside of the polymeric film, diluting and/or heating said and the contacting said delivery system with the target.

26. A method of delivering an active agent to a target according to claim 25, wherein the dilution and/or heating of the delivery system is performed simultaneous with the contacting of the delivery system with the target.

27. A method according to claim 25, wherein the active agent is a cosmetic and the target is a human body.

28. A method according to claim 25, wherein the active agent is a textile treatment agent and the target is a textile.

29. A method according to claim 25, wherein the active agent is a cleaning agent and the target is a hard surface.

30. A method according to claim 28, performed during a domestic laundering procedure.

31. A method of delivering an active agent to a target comprising an active agent encapsulated by a polymeric film comprising a hydrophobically-modified polyol, the delivery system also comprising a surfactant on the outside of the polymeric film.

32. A method of delivering an active agent to a target according to claim 31, wherein the dilution and/or heating of the delivery system is performed simultaneous with the contacting of the delivery system with the target.

33. A method according to claim 31, wherein the active agent is a cosmetic and the target is a human body.

34. A method according to claim 31, wherein the active agent is a textile treatment agent and the target is a textile.

35. A method according to claim 31, wherein the active agent is a cleaning agent and the target is a hard surface.

36. A method according to claim 34, performed during a domestic laundering procedure.
